(19) United States
(12) Patent Application Publication
(10) Pub. No.: US 2022/0112151 A1
(43) Pub. Date: Apr. 14, 2022

(54) COMPOSITIONS OF DICARBONYL SUBSTITUTED-1-ALKENE, METHODS TO MAKE THEM, POLYMERS MADE FROM THEM AND METHODS TO MAKE THE POLYMER

(71) Applicant: Sirrus, Inc., Loveland, OH (US)

(72) Inventors: Alexander Y. Polykarpov, Brunswick, OH (US); Aniruddha Palsule, Cincinnati, OH (US); Anushree Deshpande, Cincinnati, OH (US)

(21) Appl. No.: 17/405,561

(22) Filed: Aug. 18, 2021

Related U.S. Application Data
(60) Provisional application No. 63/090,507, filed on Oct. 12, 2020.

(51) Int. Cl. C07C 69/73; C07C 67/31; C08F 122/10

(52) U.S. Cl. CPC C07C 69/73; C08F 122/10; C07C 67/31

(57) ABSTRACT

A method forming a blocked 1,1-dicarbonyl substituted comprises reacting an alkene of a 1,1-dicarbonyl substituted alkene with a blocking Michael addition donor compound such as an alcohol or thiol. The blocked 1,1-dicarbonyl substituted alkene may be polymerized by providing sufficient thermal energy whereby at least portion of the blocked alkenes revert to alkenes and may be addition polymerized or Michael added with a multifunctional Michael addition donor compound (e.g., polyol or polythiol).

### FIELD

The invention relates to 1,1-dicarbonyl substituted-1-alkenes where the alkene bond has been reacted to form a blocked 1,1-dicarbonyl substituted-1-alkene. In particular, the invention relates to forming the blocked 1,1-dicarbonyl substituted-1-alkene, the composition thereof, the use of the blocked 1,1-dicarbonyl substituted-1-alkene compositions to form coatings, adhesives, articles, inks and the like.

### BACKGROUND

1,1-dicarbonyl substituted-1-alkenes have been known for some time and described in U.S. Pat. Nos. 2,330,033; 3,221,745 and 3,523,097; 3,197,318; 4,056,543 and 4,160,864. Despite this the 1,1-dicarbonyl substituted-1-alkenes were not commercialized due to the simultaneous production of detrimental by-products that resulted, for example, in stability issues and difficulty in product separation.

1,1-dicarbonyl substituted-1-alkenes compounds rapidly polymerize at room temperature under mild conditions in the presence of nucleophilic or basic initiating species, which render them both useful, as well as, present problems with their stability and workability. More recently, processes to produce the 1,1-dicarbonyl substituted-1-alkenes that solved some of the stability issues were described in U.S. Pat. Nos. 8,609,885; 8,884,405; US2014/0329980; and US 2015/0073110; all incorporated herein by reference in their entirety for all purposes. However, due to their high reactivity it is still often challenging to prepare a single component system. While curable one component compositions of methylene malonates based on latent or microencapsulated initiators have been described (e.g., U.S. Pat. No. 9,181,365) such approaches still suffer from poor compatibility with multiple pigments, fillers and other compounds due to the high reactivity of methylene malonates.

The 1,1-dicarbonyl substituted-1-alkenes have been used for certain applications. Recently, some methods were described attempting to further broaden the utility of 1,1-dicarbonylsubstituted-1-alkenes for preparation of polymers with improved properties. For example, copolymerizing two or more 1,1-dicarbonyl substituted-1-alkene monomers having substantially different homopolymer glass transition temperatures (see, for example, U.S. Pat. No. 9,315,597) have been described to produce polymers with a broad range of Tg. The 1,1-dicarbonyl substituted-1-alkenes have been reacted with diols to form polyester macromers, which are then subsequently polymerized to form coatings and the like. (U.S. Pat. No. 9,617,377). The 1,1-dicarbonyl substituted-1-alkenes have been UV polymerized with other radically polymerizable monomers and oligomers in the presence of a UV initiator (e.g., copending U.S. provisional application 62/987,507).

Accordingly, it would be desirable to provide a composition comprised of a 1,1-dicarbonyl substituted-1-alkene that enables or improves one or more characteristic such as improved stability, increased polymerization temperature, ability to deconstruct adhered substrates allowing, for example, the recycling of the adhered substrates, the adhesive and property development flexibility. Likewise, it would be desirable to enable one component systems while retaining compatibility with pigments and additives that are not currently compatible with methylene malonates.

### SUMMARY

It has been discovered that a reaction of an alkene of a 1,1-dicarbonyl substituted-1-alkene with a Michael addition donor compound (“MAD compound”) such as an alcohol or thiol by Michael addition realizes a blocked 1,1-dicarbonyl substituted-1-alkene. The blocked 1,1-dicarbonyl substituted-1-alkene may be simply heated to unblock at least a portion of the blocked alkenes of the 1,1-dicarbonyl substituted-1-alkene, which can then polymerize to form a polymer that is typically a cross-linked polymer. The polymer may form via: addition polymerization of the unblocked 1,1-dicarbonyl substituted-1-alkene, by crosslinking through multifunctional Michael addition donor compounds (e.g., polyol or polythiol) or by a combination thereof. Thus, blocking of 1,1-dicarbonyl substituted-1-alkenes via Michael addition allows for the formation of 1K curable formulations for various applications. The Michael adduct bonds may be reformed by applying heat, which may lead to new materials with reformable bonds that can render them easier to recycle or reprocess.

A first aspect of the invention is method to form a blocked 1,1-dicarbonyl substituted alkene (“blocked 1,1-dicarbonyl alkene”) comprising: mixing a 1,1-dicarbonyl substituted alkene with a blocking Michael addition donor compound, to form a reaction mixture, and providing sufficient thermal energy to the reaction mixture to react the blocking Michael addition donor with an alkene of the 1,1-dicarbonyl substituted alkene to form the blocked 1,1-dicarbonyl substituted alkene. In some instances, ambient conditions (˜20° C. to ˜25° C.) are sufficient to cause some portion of the alkenes to become blocked depending on the blocking MAD compound (e.g., methanol). Otherwise, heating above ambient temperatures may be employed. The blocking Michael addition donor compound is desirably comprised of a monofunctional Michael addition donor compound (“MAD compound”) and in one embodiment the monofunctional MAD compound is mixed with a multifunctional MAD compound. Monofunctional meaning that there is only one Michael addition donor group in the MAD compound. Multifunctional means there is more than one Michael addition donor group in the MAD compound. The blocked 1,1-dicarbonyl substituted alkene (“blocked alkene”) allows for the tailoring of polymers derived therefrom. For example, the blocked alkene may be polymerized wherein differing amounts of alkenes that become unblocked may be addition polymerized or reacted with a multifunctional Michael addition donor compounds that realize differing properties as well as subsequently modifying the properties of the polymerized article.

A second aspect of the invention is a blocked 1,1-dicarbonyl substituted alkene comprised of the reaction product of a 1,1-dicarbonyl substituted alkene and a monofunctional Michael addition donor compound. The composition of the second aspect may be further comprised of the reaction product of the 1,1-dicarbonyl substituted alkene and a multifunctional MAD compound. In an embodiment, the composition may be comprised of a monomeric or oligomeric 1,1-dicarbonyl substituted alkene (monomer meaning a monomeric 1,1-dicarbonyl substituted alkene having one alkene and oligomer meaning 2 or more monomers that have been linked as described below resulting in the oligomer having 2 or more alkenes).

A third aspect of the invention is an article comprised of a polymer formed from the blocked 1,1-dicarbonyl alkene of the first or second aspect. In an embodiment the article is comprised of the polymer disposed upon a substrate, wherein the polymer is adhered to the substrate. Such articles include, but are not limited to, substrates that have been coated or adhered to other substrates. In another embodiment, the article is an article comprised of adhered layers of the polymer such as those formed by additive manufacturing methods. In an embodiment, the monomeric 1,1-dicarbonyl substituted alkene are unblocked and the oligomer 1,1-dicarbonyl substituted alkenes are blocked, where the oligomers have at least portion of the alkenes being unblocked to form the article.

A fourth aspect of the invention is a method of deconstructing an article comprising,
(i) providing an article comprising a polymer formed from the blocked 1,1-dicarbonyl alkene of the first or second aspect,
(ii) heating the article to a deconstruction temperature, wherein the polymer is comprised of sufficient number of Michael addition crosslinks such that upon heating to the deconstruction temperature, the Michael addition crosslinks revert to alkenes allowing for the deconstruction of the article, and
(iii) deconstructing the article.

The properties of the polymer may vary widely depending on the 1,1-dicarbonyl substituted alkene used or mixtures of them used, the amount of blocking, subsequent donor compounds included when forming the polymer allowing for articles that may vary from elastic to rigid as well as have a wide range of glass transition temperatures (Tg). As such, the polymer, compositions to make polymer and articles made therefrom may be suitable for a myriad of applications such as coatings (e.g., thermally activated coatings such as can or metal coil coatings), adhesives, additive manufactured articles, molding resins, various printing inks including inkjet inks, and medical applications among others.

### DETAILED DESCRIPTION

The explanations and illustrations presented herein are intended to acquaint others skilled in the art with the invention, its principles, and its practical application. The specific embodiments of the present disclosure as set forth are not intended to be exhaustive or limit the scope of the disclosure.

One or more as used herein means that at least one, or more than one, of the recited components may be used as disclosed. It is understood that the functionality of any ingredient or component may be an average functionality due to imperfections in raw materials, incomplete conversion of the reactants and formation of by-products. The 1,1-dicarbonyl substituted-1-alkene compounds are for convenience referred to as “1,1-dicarbonyl alkene(s)” interchangeably. The blocking Michael addition donor compound is for convenience referred to as the “blocking MAD compound” or “MAD compound” depending on the context.

The method to form the blocked 1,1-dicarbonyl substituted-1-alkene compound comprises mixing a 1,1-dicarbonyl substituted-1-alkene compound with a blocking Michael addition donor compound to form a reaction mixture. The mixing may be performed under any conditions or apparatus known in the art for mixing two components. In some embodiments, a solvent may be used to dissolve the 1,1-dicarbonyl substituted alkene and MAD compound to form the mixture. Useful solvents may include any solvent that dissolves the 1,1-dicarbonyl substituted alkenes and the MAD compound if necessary. Solvents may, for example, be any polar solvent, a protic solvent, aromatic hydrocarbon, water or combination thereof. Solvents may, for example, include aromatic hydrocarbons, ethers, ketones and the like so long as they are not a MAD compound or interfere with the blocking of the 1,1-dicarbonyl substituted alkenes.

The reaction mixture is then reacted to cause at least a portion of the alkenes of the “1,1-dicarbonyl alkene(s)” to react with the blocking MAD compound. To reiterate, depending on the blocking MAD compound ambient conditions may be enough to block at least a portion of alkenes of the 1,1-dicarbonyl alkene. Typically, however, the reaction mixture is heated to a reaction temperature above ambient such as 40° C., 60° C., 80° C. or 100° C. to 120° C., 140° C. and about 150° C. for any practicable time useful to make the blocked 1,1-dicarbonyl alkene from several minutes to several days. The temperature may be below the boiling temperature of the MAD compound or above. And, when above, reflux may be employed. The reaction may be performed at any practicable pressure such as ambient pressures and desirably under reflux (e.g., to shorten the time). The pressure may also be above atmospheric pressure to minimize volatility, for example, of a low molecular weight blocking MAD compound.

In carrying out the reaction, the amount of alkenes that are blocked may be any sufficient to realize one or more of the desirable characteristics mentioned above. Typically, the amount of alkenes that are blocked are at least 25%, 35%, 50% to 80%, 90%, 95% or essentially all percent by mole.

Herein, the blocked 1,1-dicarbonyl substituted-1-alkene compound is understood to be insufficiently cross-linked so that it is still usable to formulate adhesives, coatings and the like. Generally, this means the blocked 1,1-dicarbonyl substituted-1-alkene compound is a liquid at ambient conditions or may be heated below the reaction temperature to form a liquid. Typically, the amount of blocking and or use of monofunctional blocking MAD compounds may be used concurrently or alone to realize a liquid or solid blocked 1,1-dicarbonyl substituted-1-alkene compound. Typically, a blocked 1,1-dicarbonyl substituted-1-alkene compound exhibits a viscosity, which facilitates formulation of a mixture useful to make coatings, adhesives, caulks, molded articles and additive manufactured articles. Typically, the viscosity of the formulated mixture is at most about 100,000 centipoise (cps), 50,000 cps, 30,000 to about 1, 10, 100 or 1000 cps at a shear rate used to dispose, pump or mold an article from the formulated mixture. The rheological behavior may be Newtonian or non-Newtonian and in some instances display a yield point (e.g., when formulated into a mixture with other ingredients).

The completeness of blocking of the carbon-carbon double bonds may be determined using quantitative HNMR (hydrogen nuclear magnetic resonance), Fourier transform infrared spectroscopy or any other method that detects the disappearance of carbon-carbon double bonds.

The 1,1-dicarbonyl alkenes are compounds wherein a central carbon atom is doubly bonded to another carbon atom to form a double bond. The central carbon atom is further bonded to two carbonyl groups. Each carbonyl group is bonded to a hydrocarbyl group either directly or through an oxygen atom. Where the hydrocarbyl group is bonded to the carbonyl group, a ketone group is formed. Where the hydrocarbyl group is bonded to the carbonyl group through an oxygen atom, an ester group is formed. The 1,1-dicarbonyl alkene may have a structure as shown below in Formula I, where X¹ and X² are an oxygen atom or a direct bond, and where R¹ and R² are each hydrocarbyl groups that may be the same or different. Both X¹ and X² may be oxygen atoms, such as illustrated in Formula IIA, one of X¹ and X² may be an oxygen atom and the other may be a direct bond, such as shown in Formula IIB, or both X¹ and X² are direct bonds, such as illustrated in Formula IC. The 1,1-dicarbonyl alkene compounds used herein may have all ester groups (such as illustrated in Formula IIA), all keto groups (such as illustrated in Formula IIC) or a mixture thereof (such as illustrated in Formula IIB). Compounds with all ester groups may be preferred in some applications due to the flexibility of synthesizing a variety of such compounds.

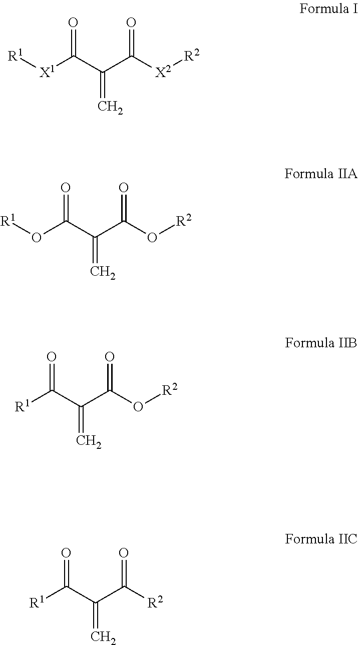

Hydrocarbyl as used herein refers to a group containing one or more carbon atom backbones and hydrogen atoms, which may optionally contain one or more heteroatoms. Heteroatom means nitrogen, oxygen, sulfur and phosphorus, more preferred heteroatoms include nitrogen and oxygen. Where the hydrocarbyl group contains heteroatoms, the heteroatoms may form one or more functional groups well known to one skilled in the art. Hydrocarbyl groups may contain cycloaliphatic, aliphatic, aromatic or any combination of such segments. The aliphatic segments can be straight or branched. The aliphatic and cycloaliphatic segments may include one or more double and/or triple bonds. Included in hydrocarbyl groups are alkyl, alkenyl, alkynyl, aryl, cycloalkyl, cycloalkenyl, alkaryl and aralkyl groups. Cycloaliphatic groups may contain both cyclic portions and noncyclic portions. Hydrocarbylene means a hydrocarbyl group or any of the described subsets having more than one valence, such as alkylene, alkenylene, alkynylene, arylene, cycloalkylene, cycloalkenylene, alkarylene and aralkylene. One or both hydrocarbyl groups may consist of one or more carbon atoms and one or more hydrogen atoms. As used herein percent by weight or parts by weight refer to, or are based on, the weight of the solution composition unless otherwise specified.

A preferred class of 1,1-dicarbonyl alkene compounds is methylene malonates, the core structural unit/formula for which is shown below:

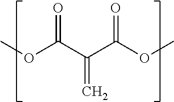

The term “monofunctional” refers to 1,1-dicarbonyl alkene compounds or a methylene malonate having only one core unit or one carbon-carbon double bond. The term “difunctional” refers to 1,1-dicarbonyl alkene compounds or a methylene malonate having two core unit bound through a hydrocarbyl linkage between one oxygen atom on each of two core formulas. The term “multifunctional” refers to 1,1-dicarbonyl alkene compounds or methylene malonates having more than one core formula which forms a chain through a hydrocarbyl linkage between one oxygen atom on each of two adjacent core formulas.

The hydrocarbyl groups (e.g., R¹ and R²), each may comprise straight or branched chain alkyl, straight or branched chain alkyl alkenyl, straight or branched chain alkynyl, cycloalkyl, alkyl substituted cycloalkyl, aryl, aralkyl, or alkaryl. The hydrocarbyl group may optionally include one or more heteroatoms in the backbone of the hydrocarbyl group. The hydrocarbyl group may be substituted with a substituent that does not negatively impact the ultimate function of the 1,1-dicarbonyl alkene or the polymer prepared from the 1,1-dicarbonyl alkene. Preferred substituents include alkyl, halo, alkoxy, alkylthio, hydroxyl, nitro, cyano, azido, carboxy, acyloxy, and sulfonyl groups. More preferred substituents include alkyl, halogen, alkoxy, allylthio, and hydroxyl groups. Most preferred substituents include halogen, alkyl, and alkoxy groups.

As used herein, alkaryl means an alkyl group with an aryl group bonded thereto. As used herein, aralkyl means an aryl group with an alkyl group bonded thereto and include alkylene bridged aryl groups such as diphenyl methyl groups or diphenyl propyl groups. As used herein, an aryl group may include one or more aromatic rings. Cycloalkyl groups include groups containing one or more rings, optionally including bridged rings. As used herein, alkyl substituted cycloalkyl means a cycloalkyl group having one or more alkyl groups bonded to the cycloalkyl ring.

The hydrocarbyl groups may include 1 to 30 carbon atoms, 1 to 20 carbon atoms, or 1 to 12 carbon atoms. Hydrocarbyl groups with heteroatoms in the backbone may be alkyl ethers having one or more alkyl ether groups or one or more alkylene oxy groups. Alkyl ether groups may be ethoxy, propoxy, and butoxy. Such compounds may contain from about 1 to about 100 alkylene oxy groups, about 1 to about 40 alkylene oxy groups, about 1 to about 12 alkylene oxy groups, or about 1 to about 6 alkylene oxy groups.

One or more of the hydrocarbyl groups (e.g., R¹, R², or both) may include a C₁-C₁₅ straight or branched chain alkyl, a C₁-C₁₅ straight or branched chain alkenyl, a C₅-C₁₈ cycloalkyl, a C₆-C₂₄ alkyl substituted cycloalkyl, a C₄-C₁₈ aryl, a C₄-C₂₀ aralkyl, or a C₄-C₂₀ aralkyl. The hydrocarbyl group may include a C₁-C₈ straight or branched chain alkyl, a C₅-C₁₂ cycloalkyl, a C₆-C₁₂ alkyl substituted cycloalkyl, a C₄-C₁₈ aryl, a C₄-C₂₀ aralkyl, or a C₄-C₂₀ aralkyl.

Alkyl groups may include methyl, propyl, isopropyl, butyl, tertiary butyl, hexyl, ethyl pentyl, and hexyl groups. More preferred alkyl groups include methyl and ethyl. Cycloalkyl groups may include cyclohexyl and fenchyl. Alkyl substituted groups may include menthyl and isobornyl, norbornyl as well as any other bicyclic, tricyclic or polycyclic structure.

Hydrocarbyl groups attached to the carbonyl group may include methyl, ethyl, propyl, isopropyl, butyl, tertiary, pentyl, hexyl, octyl, fenchyl, menthyl, and isobornyl, cyclic, bicyclic or a tricyclic group such as cyclohexyl, norbornyl, or tricyclodecanyl.

The 1,1-dicarbonyl alkene may be comprised of one or more of methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

Some or all of the 1,1-dicarbonyl alkene can also be multifunctional, having more than one core unit and thus more than one alkene group. Exemplary multifunctional 1,1-dicarbonyl alkenes are illustrated by the formula:

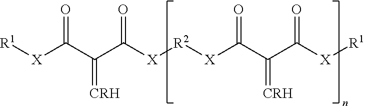

wherein R¹ and R² are as previously defined; X is, separately in each occurrence, an oxygen atom or a direct bond; n is an integer of 1 or greater to any useful amount such as a polymer of 1,000 or 10,000 Daltons to about 1,000,000 or 100,000 and R is hydrogen or a hydrocarbyl group having 1 to 30 carbons, so long as at least one R is hydrogen (i.e., ═CH₂) and preferably every R is hydrogen. Typically, n is 1 or 2 to 20 or 10.

In exemplary embodiments R² may be, separately in each occurrence, straight or branched chain alkyl, straight or branched chain alkenyl, straight or branched chain alkynyl, cycloalkyl, alkyl substituted cycloalkyl, aryl, aralkyl, or alkaryl, wherein the hydrocarbyl groups may contain one or more heteroatoms in the backbone of the hydrocarbyl group and may be substituted with a substituent that does not negatively impact the ultimate function of the compounds or polymers prepared from the compounds. Exemplary substituents may be those disclosed as useful with respect to R¹. In certain embodiments R² may be, separately in each occurrence, C₁₋₁₅ straight or branched chain alkyl, C₂₋₁₅ straight or branched chain alkenyl, C₅₋₁₈ cycloalkyl, C₆₋₂₄ alkyl substituted cycloalkyl, C₄₋₁₈ aryl, C₄₋₂₀ aralkyl or C₄₋₂₀ aralkyl groups. In certain embodiments R² may be separately in each occurrence C₁₋₈ straight or branched chain alkyl, C₅₋₁₂ cycloalkyl, C₆₋₁₂ alkyl substituted cycloalkyl, C₄₋₁₈ aryl, C₄₋₂₀ aralkyl or C₄₋₂₀ alkaryl groups.

In an embodiment, X is O and R² is the residue of a diol, wherein a polyester is formed. The polyesters may be formed from any suitable 1,1-dicarbonyl alkene such as the malonates described above and as described in U.S. Pat. No. 9,969,822 from col. 19, line 49 to col. 20, line 3 and a polyol incorporated herein by reference. Examples of suitable polyols include, for example, those described in U.S. Pat. No. 9,969,822 from col. 20, line 18 to col. 21, line 26, incorporated herein by reference. Examples of diols may include ethylene diol 1,3-propylene diol, 1,2 propylene diol, 1-4-butanediol, 1,2-butane diol, 1,3-butane diol, 2,3-butane diol, 1,5-pentane diol, 1,3- and 1,4-cyclohexanedimethanols or combinations thereof. Examples of triols may include 1,2,3-propane triol, 1,2,3-butane triol, trimethylolpropane, 1,2,4-butane triol or combination thereof. Likewise, the polyol may be even higher functional, for example, di(trimethylolpropane), pentaerythritol, dipentaerythritol or combination thereof. Any combination of polyols such as multiple diols, triols, tetraols, pentaols, hexaols or mixtures thereof may be used.

The 1,1-dicarbonyl alkene may be produced and purified by the methods described in U.S. Pat. Nos. 8,609,8985; 8,884,051; 9,108,914 and 9,518,001 and Int. Pub. WO 2017/197212. Examples of such 1,1-dicarbonyl alkenes are available under the tradenames CHEMILIAN and FORZA and include, for example, methylene malonate, dihexyl methylene malonate, dicyclohexyl methylene malonate and multifunctional polyester methylene malonates available from Sirrus, Inc., Loveland, Ohio (Nippon Shokubai, Japan).

### Michael Addition Donor Compounds (“MAD Compound”)

The MAD compound is comprised of a Michael addition donor group such as an amine, alcohol, carboxylic acid or thiol. The MAD compound may be monofunctional (only one Michael addition group—“MAD group”) or multifunctional (2 or more MAD groups). The MAD compound may contain a mixture of alcohol, carboxyl and thiol groups. Desirably, the MAD group is a thiol or alcohol or mixture thereof.

Because amines may cause anionic polymerization of the alkenes of the 1,1-dicarbonyl alkenes, the amine blocking MAD compound requires significant dilution to avoid anionic polymerization to realize the blocked 1,1-dicarbonyl alkene. The amine blocking MAD compound may be a primary or secondary amine. Generally, this requires that the amine MAD compound is mixed with the 1,1-dicarbonyl alkenes being diluted using a solvent. The excess solvent can then be removed by distillation using various techniques with or without vacuum.

The MAD compound may be a small molecule, an oligomer, or polymer having a Michael addition donor group. The MAD groups may be pendant from the polymer backbone, at the terminus of the backbone whether linear or branched or any combination thereof including dendritic. The MAD compound may be a mixture of monofunctional and multifunctional MAD compounds. The multifunctional MAD compound typically has from 2 to 10, 8, 5 or 4 Michael addition donor groups. The average functionality of the mixture of MAD compounds may be from 1 or above 1, 1.5 or 2 to about 10, 8, 5 or 4.

The MAD compound may be an oligomer or polymer of a polyolefin, polyether, polyester, acrylic (acrylic polyols), polycarbonate (e.g., polycarbonate polyol), epoxies, carboxylic acid functional polyesters and acrylics or any mixture or combination thereof. The molecular weight of the MAD compound may vary of over a wide range, for example, from 30 g/moles to a million g/moles weight average molecular weight (Mw). In one embodiment the MAD has a Mw of at most about 500 g/moles, 200 g/moles, or even 150 g/moles. In another embodiment, the MAD compound is comprised of a polymer or oligomer having an Mw of at least 500, 1000 or 5000 g/moles to about 1,000,000, 500,000, 200,000, 100,000, 50,000 or 10,000 g/moles. In another embodiment, the reaction mixture to form the blocked 1,1-dicarbonyl alkene may contain a low molecular weight monofunctional MAD compound and a multifunctional compound having a high Mw, where low Mw is less than or equal to about 500 g/moles and high Mw is greater than about 500 g/moles, 1000 g/moles or 5000 g/moles.

In an embodiment the MAD compound is comprised of a MAD compound having a Mw of less than 500, 200 or 150 g/moles. It may be desirable to have a low Mw for the MAD compound to aid in removal of the MAD compound on polymerizing the blocked 1,1-dicarbonyl alkene. Likewise, it may be desirable to have the low molecular MAD compound be monofunctional. In this embodiment, it may be desirable for the MAD compound to be an alcohol or thiol. Exemplary alcohols for this embodiment may be, methanol, ethanol, propanol, butanol, pentanol, hexanol, heptanol, octanol or mixture thereof or their analogous thiols as well as thioterpineol, thioacetic acid, mercaptoethanol or mixtures thereof.

In some instances it may be desirable for there to be 2 or more MAD groups when the MAD compound is an oligomer or polymer. The chemistry of the backbone may impart other desired properties such as dispersability or solubulization of the blocked 1,1-dicarbonyl substituted-1-alkene compound or making it more compatible with other ingredients in the composition described herein. For example, the MAD compound may be an alkoxylated alcohol (i.e., polyether backbone) that improves dispersability in water such as those described below. The polymer or oligomer is desirably soluble, for example, to realize homogeneous distribution, in the 1,1-dicarbonyl substituted alkenes at the concentration wanted to realize the intended blocked 1,1-dicarbonyl substituted alkene and polymer formed therefrom. The polymer or oligomer MAD compound may have an alkenyl group (e.g., from about 1 to 5 or 4 alkenyl groups per molecule/chain).

The blocking MAD compound may be a carboxylic acid, amine, hydroxyl or thiol containing polyolefin, polyether, polyester, polyether-ester, polyamide, polyurethane, silicone, polystyrene, rubber including a core shell rubber, a carboxylic acid (e.g., naturally occurring carboxylic acid), fatty acid toll oil fatty acids or any combination thereof. As stated above, the blocking MAD compound may be monofunctional or multifunctional such as multifunctional amines or amine alkoxylates.

The blocking MAD compound may be any suitable polyol such as those known in the art (e.g., in the polyurethane art) such as a polyether polyol, polyester polyol or combination thereof. Exemplary polyols are described U.S. Pat. No. 5,922,809 at column 4, line 60 to column 5, line 50, incorporated herein by reference.

The acids, thiols or alcohols may be saturated or unsaturated. The natural occurring carboxylic acid maybe any natural occurring carboxylic acid, for example, abietic acid, gallic acid, azelaic acid, caffeic acid, malic acid, pyruvic acid, niacin, citric acid, biotin, abietic acid, cholic resin, pectin, alginic acid, gum rosin (a mixture of naturally occurring natural acids) or combination thereof. The fatty acid may be any fatty acid derived from any animal fat or vegetable oil and maybe saturated or unsaturated. Exemplary oils include linseed, palm, coconut, palm, olive, tung, soybean, peanut, sunflower, cotton seed, rapeseed, or combination thereof. Any fatty acid derived from the aforementioned oils and fats may be used. In an embodiment, the fatty acid is dimerized to form a dimer, trimer acid or higher polymeric acids. Typically, readily available dimer acids contain some small fraction of monomeric acid, trimer acid and higher polymeric acid. Such Dimer acids are readily available, for example, from Oleon Nev. Ertevelde, Belgium under the tradename RADIACID. In another embodiment, an unsaturated oil may be grafted with maleic anhydride at the unsaturated bond to form a pendant anhydride, which may then be further reacted to form a carboxylic acid group.

The blocking MAD compound may be any alcohol, glycol, thiol or dithiol, carboxylic acid such as carboxylic acids or dicarboxylic acids having linear or branched alkane chains of 3 or 5 to 30, 20 or 15 carbons or mixtures thereof. Examples may include hexanoic acid, hexanedioic acid, heptanoic acid, heptanedioic acid, octanoic acid, octanedioic acid, nonanoic acid, nonanedioic acid, decanoic acid, decanedioic acid, 2,3-dimethylbutanedioic, 2,2-dimethylbutanedioic acid, 3-methylheptanedioic acid, their analogous alcohols or thiols or mixtures thereof.

The blocking MAD compound may be an amine or carboxyl terminated polybutadiene, amine or carboxyl terminated polyisoprene, amine or carboxyl terminated copolymer of butadiene and acrylonitrile (CTBN), or combination thereof. The MAD compound may be an amine or carboxyl terminated homopolymer of a conjugated diene or copolymer of a conjugated diene, especially a diene/nitrile copolymer. The conjugated diene is preferably butadiene or isoprene, with butadiene being especially preferred. When a copolymer with the conjugated diene is employed with a nitrile monomer, the nitrile monomer is desirably acrylonitrile. Preferred copolymers are butadiene-acrylonitrile copolymers. The blocking MAD compound of conjugated diene copolymers contain, in the aggregate, no more than about 30 wt % of polymerized unsaturated nitrile monomer, and preferably no more than about 26 wt % of polymerized nitrile monomer. These conjugated dienes and copolymers thereof generally contain from about 1.5, more preferably from about 1.8, to about 2.5, more preferably to about 2.2 carboxyl groups per molecule, on average.

Suitable amine or carboxyl-functional butadiene and butadiene/acrylonitrile copolymers are commercially available from CVC Thermoset Specialties as Hypro® 2000×162 carboxyl-terminated butadiene homopolymer and Hypro® 1300×31, Hypro® 1300×8, Hypro® 1300×13, Hypro® 1300×9 and Hypro® 1300×18 carboxyl-terminated butadiene/acrylonitrile copolymers. The molecular weight (Mw) of these copolymers are typically from about 2000 to about 6000, more preferably from about 3000 to about 5000.

In a particular embodiment, the blocking MAD compound may be a core shell rubber. Any core shell rubber having, for example, amine or carboxyl groups or other MAD group in or on the shell (polymer of the shell contains carboxyl) of the core shell rubber particle may be used such as described in U.S. Pat. No. 4,315,085. The core-shell rubber component is a particulate material having a rubbery core. Illustrative core-shell rubber compositions are disclosed in U.S. Pat. No. 7,625,977. The rubbery core of such core shell rubber may have a Tg of less than −25° C., more preferably less than −50° C., and even more preferably less than −70° C. The Tg of the rubbery core may be well below −100° C. The core-shell rubber also typically has at least one shell portion that preferably has a Tg of at least 50° C. By “core,” it is meant an internal portion of the core-shell rubber. The core may form the center of the core-shell particle, or an internal shell or domain of the core-shell rubber. A shell is a portion of the core-shell rubber that is exterior to the rubbery core. The shell portion (or portions) typically forms the outermost portion of the core-shell rubber particle. The shell material is preferably grafted onto the core or is cross-linked. The rubbery core may constitute from 50 to 95%, especially from 60 to 90%, of the weight of the core-shell rubber particle. The molecular weight average Mw of the shell polymer may be from 10,000 to 500,000 g/moles.

The blocking MAD compound may be a MAD group terminated ester that is solid or liquid. Exemplary polyesters include polyesters obtained by the reaction of acids with epoxies such as described by WO1998023661A1. The polyester may be one that is linear terminated with a diol that is reacted with trimellitic anhydride to acid functionalize the polyester. The acid functional polyester may be a solid acid terminated polyester such as described by US Pat. App. No. 2007/0260003. An acid functional polyester polyol may also be used as the blocking MAD compound, such as those commercially available under the tradename DICAP from Geo Specialty Chemicals, Ambler, Pa. An alkyd (e.g., reaction product of a polybasic, a polyhydric alcohol, fatty acids, multifunctional fatty acids and anhydrides) having unreacted carboxylic acid groups may also be used. Another example of a suitable polyester are polyester resins available under the tradename DYNAPOL from Evonik Corp. Parsippany, N.J. The alkyd may be a long oil, medium oil, or short oil alkyd resin such as those commercially available from Deltech Corp. Baton Rouge, La.

The blocking MAD compound may also be a carboxyl functional polyurethane such as those used to make polyurethane dispersions prior to being neutralized, for example, with a base. Examples of such carboxyl terminate polyurethanes that may be useful include, for example, those described in U.S. Pat. Nos. 5,473,011 and 7,589,148.

The blocking MAD compound may be a polyolefin containing an amine, hydroxyl, carboxyl or thiol groups such those known in the art. Examples of these may include ethylene (meth)acrylic acid copolymers such as those available from DuPont under the tradename NUCREL, The Dow Chemical Company under the tradename PRIMACOR and ExxonMobil under the tradename ESCOR. Other examples may include acid grafted polyolefins (e.g., polyethylene, polypropylene that are linear, branched, or copolymers with alkenes having more than 3 carbons to 20 carbons). Examples of such grafted polyolefins may include maleic anhydride grafted polyethylenes available from The Dow Chemical Company under the tradename AMPLIFY GR.

The blocking MAD compound may be any suitable polystyrene containing a MAD group such as those known in the art including, for example, copolymers of styrene and maleic anhydride. Examples of polystyrene containing a carboxyl or anhydride may include copolymers of styrene and maleic anhydride, which may include other copolymers. Suitable such copolymer may include those commercially available from Polyscope Polymer BV under the tradename XIRAN.

The blocking MAD compound may a silicone containing a MAD group such as those known in the art including, for example, carboxyl and OH, SH terminated silicone fluids available from Momentive under the tradename MAGNASOFT and carboxyl terminated polydimethylsiloxane available from Gelest Inc. under product code DMS-B25, DMS-B31, DMS B12 and the like.

In an embodiment, the MAD group such as a carboxyl group may be obtained by hydrolysis of corresponding ester groups followed by neutralization of the formed salts with a strong acid or ion exchange and then reacting to form the blocked 1,1-dicarbonyl alkene. For example, the blocking MAD compound may be a copolymer of a methylene malonate and a vinyl acetate as described by Matsumura and Tanaka, Journal of Environmental Polymer Degradation, Vol. 2, No. 2. 1994, pp 89-97, illustrated by:

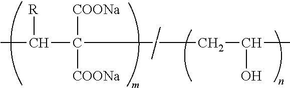

where R is H or a hydrocarbyl group and m and an n may be any useful integers so long as m+n is greater than 2 to typically 1000 or 100, wherein the salt may be, ion exchanged or hydrolyzed and be used to block the 1,1-dicarbonyl alkene.

In an embodiment, the method makes a blocked 1,1-dicarbonyl alkene that is comprised of the reaction product of a blocked 1,1-dicarbonyl alkene and a monofunctional blocking MAD compound such as an amine, thiol, carboxylic acid or alcohol and desirably an alcohol. The blocked 1,1-dicarbonyl alkene may be further comprised of the reaction product of the 1,1-dicarbonyl substituted alkene and a multifunctional blocking MAD compound. The blocked 1,1-dicarbonyl substituted alkene may also be further comprised of a strong acid that is separately added, which may stabilize any remaining alkenes from addition polymerizing. The strong acid may be any inorganic acid or organic acid, for example that has a pKa of about −12 to 3. Examples of such acids include methane sulfonic acid or toluene sulfonic acid. Desirably, the 1,1-dicarbonyl substituted alkene is a polyester oligomer described above. The average functionality (alkene per molecule) is generally from essentially 0 to 10, 5, 2, 1 or less than 1. The monofunctional blocking MAD compound desirably has a molecular weight of at most 500 g/moles. The blocked 1,1-dicarbonyl substituted alkene may also have some unblocked alkenes such as 1%, 5% or even 10% by mole unblocked alkenes.

The blocked 1,1-dicarbonyl substituted alkene may have further components added to it that may vary depending on the application. The further components may be one or more dyes, pigments, anticorrosive pigments, toughening agents, rheology modifiers, fillers, reinforcing agents, thickening agents, opacifiers, inhibitors, fluorescence markers, thermal degradation reducers, thermal resistance conferring agents, surfactants, wetting agents, defoamers, flow or leveling agents, pigment extenders, biocides, scratch and abrasion resistance additives, slip and release aids, surface texture and appearance enhancing additives, matting agents, anti-graffiti agents, antifouling additives, adhesion promoting additives, thermal conductive agents, or stabilizers can be included the composition. In certain embodiments, such thickening agents and other compounds can be used to increase the viscosity of a polymerizable system comprised of the blocked 1,1-dicarbonyl substituted alkene from about 1 to 3 cPs to about 30,000 cPs, or more.

In an embodiment, the blocked 1,1-dicarbonyl substituted alkene is used to make a polymer, which may be linear, branched, dendritic, or cross-linked, comprising shaping the blocked 1,1-dicarbonyl substituted alkene and heating the provided 1,1-dicarbonyl substituted alkene. The heating is to a polymerization temperature wherein at least a portion of the blocked alkenes of the blocked 1,1-dicarbonyl substituted alkene become unblocked (i.e., revert to alkenes) and polymerization of the unblocked 1,1-dicarbonyl substituted alkene proceeds through addition polymerization of the unblocked alkenes, Michael addition of the unblocked alkenes or combination thereof.

The polymerization temperature maybe any useful to unblock a desired proportion of blocked alkenes in the blocked 1,1-dicarbonyl substituted alkene and initiate the desired polymerization. Generally, the temperature is above ambient to about the decomposition temperature of the underlying blocked 1,1-dicarbonyl substituted alkene. Typically the temperature is from about 40° C., 60° C., 80° C. or 100° C., 120° C. to about 250° C., 200° C., 180° C., 150° C., or 140° C. In a particular embodiment the blocked alkene is used as a powder coating that may be polymerized at the aforementioned temperatures (e.g., low bake: 120° C. to 140° C. or standard bake: 150° C. to 180° C.).

In an embodiment, the blocking MAD compound is removed during polymerizing due its volatility at the polymerization temperature at ambient pressures (evaporates). The removal may be facilitated by polymerizing under flowing gas (e.g., air) or a vacuum at the polymerization temperature. The vacuum may be any useful such as just below 1 atmosphere to 0.0001, 0.01 or 0.1 atmosphere. The volatile blocking MAD compound may be used to assist in forming a foam if desired. The volatile MAD compound generally has an MW as described previously for low Mw blocking MAD compounds. Desirably the volatile blocking MAD compound is monofunctional as well as being an alcohol, thiol or mixture of these with alcohols being particularly suitable that desirably are of a low molecular weight. In another embodiment, a blocked 1,1-dicarbonyl substituted alkene that has been blocked with a monofunctional amine MAD compound is mixed with other blocked 1,1-dicarbonyl substituted alkenes and then heated to unblock the blocked 1,1-dicarbonyl substituted alkene and polymerize the mixture. The amine blocked 1,1-dicarbonyl substituted alkene may be desirable to accelerate the anionic initiation of addition polymerization of a blocked 1,1-dicarbonyl substituted not blocked with an amine as they become unblocked upon heating as well as the amine blocked 1,1-dicarbonyl substituted alkenes.

The blocked 1,1-dicarbonyl substituted alkene may be further mixed with another multifunctional Michael addition donor compound that may react to crosslink the blocked 1,1-dicarbonyl substituted alkene as it becomes unblocked. These cross-linking multifunctional MAD compounds typically will have a higher Mw as described above, which typically is higher than the Mw of the blocking MAD compound. The particular cross-linking MAD compound may be selected on desired properties of the end application such as the multifunctional blocking MAD compounds described above.

The polymer formed by polymerizing by unblocking the blocked 1,1-dicarbonyl substituted alkene may be comprised of essentially addition polymerized unblocked alkene bonds or cross-linking through Michael addition by multifunctional MAD compounds. Typically, there is a proportion of Michael addition adducts and addition polymerization of the alkenes. For example, at least 10%, 20% or even 50% of the alkenes (i.e., mole percent) may be Michael addition adducts or vice versa at least 10%, 20% or even 50% of the alkenes undergo addition polymerization to form the polymer. The amount of addition polymerization or polymerization by Michael addition may be determined by weight loss by known methods such as thermogravimetric analysis (TGA).

If it is desired to have the blocked 1,1-dicarbonyl substituted alkene to undergo addition polymerization, an anionic initiator and desirably a latent initiator may be added to the blocked 1,1-dicarbonyl substituted alkene prior to or during the unblocking and polymerization. The anionic initiator may be any suitable anionic initiator that initiates anionic polymerization upon contact with the unblocking 1,1-dicarbonyl substituted alkenes.

A wide variety of anionic initiators may be used including most nucleophilic initiators capable of initiating anionic polymerization. Exemplary initiators include alkali metal salts, alkaline earth metal salts, ammonium salts, amines, halides (halogen containing salts), metal oxides, and mixtures containing such salts or oxides. Exemplary anions for such salts include anions based on halogens, acetates, benzoates, sulfur, carbonates, silicates and the like. Examples of anionic initiators may include glass beads having a basic constituent such as soda-lime silica glass, ceramic beads (comprised of various metals, nonmetals and metalloid materials), clay minerals (including hectorite clay and bentonite clay), and ionic compounds such as sodium silicate, sodium benzoate, and calcium carbonate. Additional suitable anionic initiators are also disclosed in U.S. Pat. No. 9,181,365 (col. 9, lines 45-57) and U.S. Pat. No. 9,334,430 incorporated herein by reference.

The amine anionic initiator may be any primary, secondary or tertiary amine and may be an alkyl or substituted alkyl amine. Desirably, the amine is one that does not substantially volatilize at the polymerization temperature. As described above, the amine initiator may arise from amine blocked 1,1-dicarbonyl substituted alkene. The alkyl or substituted alky may be any hydrocarbyl group typically having from 1 to 30 carbons and 1 to 6 heteroatoms such as sulfur and oxygen. In an embodiment the amines include, for example, substituted (e.g., 1 or 2 heteroatoms) or unsubstituted C₁-C₅ mono- and diamines, aromatic amines, and mixtures thereof. The amine compounds may have molecular weights from about 50 to about 10,000. In general, the lower molecular weight amines may be desired, for example, to enhance ease of mixing. Lower molecular weight amines generally have a molecular weight of less than about 1500 g/moles or less than about 1000 g/mol. Examples of amines include ethylamine, diethylamine, triethylamine, ethanolamine, diethanolamine, triethanolamine, butylamine, dibutylamine, tributylamine, butanolamine, dibutanolamine, tributanolamine, propanolamine, dipropanolamine, tripropanolamine, propylamine, dipropylamine, tripropylamine, ethylenediamine, triethylenediamine, N,N-dimethylbenzylamine, isophoronediamine, ethyl 1-methyl-3-piperidinecarboxylate, ethyl-1-methyl-4-piperidinecarboxylate, bis(2,2-morpholylethyl)ether (DMDEE), and mixtures thereof.

The anionic initiator may be employed in any amount sufficient to facilitate the anionic polymerization to the extent desired. The anionic initiator typically is used in an amount of about 0.01 to 20%, or about 0.1 or 0.25 to 8%, of about 0.5 to 5% or about 0.75 to 2% by weight of 1,1-dicarbonyl substituted alkene or the 1,1-dicarbonyl substituted alkene and any other component mixed with the 1,1-dicarbonyl substituted alkene.

In some embodiments the anionic initiator may be latent to assist in attaining the desired ratio of addition polymerization and Michael addition crosslinking, wherein each of the components to be mixed with the 1,1-dicarbonyl substituted alkene is contained in one package together that is activated, for example, upon discharge from the package and application of some force. For example, the composition may be discharge and mixed by a static or dynamic mixer activating the latent initiator. In other embodiments the anionic initiator may be activated by irradiating, heating or exposing to a solvent, for example that dissolves a coating or encapsulant enveloping the initiator.

The blocked 1,1-dicarbonyl substituted alkene may be contained separately and brought together and mixed with other ingredients (e.g., anionic initiator, multifunctional crosslinking MAD compound, fillers dyes and the like) to polymerize the blocked 1,1-dicarbonyl substituted alkene to form the polymer. Suitable separate packaging or delivery systems may include those known in the art. Illustrative examples include those involving separate rigid tubes in which each material is dispensed by a separate plunger and mixed upon exiting using a static or dynamic mixing nozzles such as described by Craig Blum, Two Component Adhesive Cartridge Systems, FAST, July 2008. In another illustration, two or more compartmented sausage containers may be used such as described in U.S. Pat. Nos. 4,009,778; 4,126,005; 4,227,612; 6,129,244; 8.313,006 and 9,821,512.

The latent anionic initiator may be any employing an encapsulant. An illustrative example those described in U.S. Pat. No. 9,334,430 incorporated herein by reference in its entirety for all purposes.

In another embodiment, the anionic initiator may be a latent base such as those that absorb radiation such as radiation in the UV or visible region and forms a base or nucleophilic anionic initiator

Examples of photolatent bases include photocleavable carbamates (e.g., 9-xanthenylmethyl, fluorenylmethyl, 4-methoxyphenacyl, 2,5-dimethylphenacyl, benzyl, and others), which have been shown to generate primary or secondary amines after photochemical cleavage. Other photolatent bases which generate primary or secondary amines include certain O-acyloximes, sulfonamides, and formamides. Acetophenones, benzophenones, and acetonaphthones bearing quaternary ammonium undergo photocleavage to generate tertiary amines in the presence of a variety of counter cations (borates, dithiocarbamates, and thiocyanates). Examples of these photolatent ammonium salts are N-(benzophenonemethyl)tri-N-alkyl ammonium tetraarylborates or alkyltriarylborates or dialkyldiarylborates. Sterically hindered α-aminoketones generate tertiary amines. Exemplary photolatent bases useful for practicing the present disclosure include 5-benzyl-1,5-diazabicyclo[4.3.0]nonane, 5-(anthracen-9-yl-methyl)-1,5-diaza[4.3.0]nonane, 5-(2′-nitrobenzyl)-1,5-diazabicyclo[4.3.0]nonane, 5-(4′-cyanobenzyl)-1,5-diazabicyclo[4.3.0]nonane, 5-(3′-cyanobenzyl)-1,5-diazabicyclo[4.3.0]nonane, 5-(anthraquinon-2-yl-methyl)-1,5-diaza[4.3.0]nonane, 5-(2′-chlorobenzyl)-1,5-diazabicyclo[4.3.0]nonane, 5-(4′-methylbenzyl)-1,5-diazabicyclo[4.3.0]nonane, 5-(2′,4′,6′-trimethylbenzyl)-1,5-diazabicyclo[4.3.0]nonane, 5-(4′-ethenylbenzyl)-1,5-diazabicyclo[4.3.0]nonane, 5-(3′-trimethylbenzyl)-1,5-diazabicyclo[4.3.0]nonane, 5-(2′,3′-dichlorobenzyl)-1,5-diazabicyclo[4.3.0]nonane, 5-(naphth-2-yl-methyl-1,5-diazabicyclo[4.3.0]nonane, 1,4-bis(1,5-diazabicyclo[4.3.0]nonanylmethyl)benzene, 8-benzyl-1,8-diazabicyclo[5.4.0]undecane, 8-benzyl-6-methyl-1,8-diazabicyclo[5.4.0]undecane, 9-benzyl-1,9-diazabicyclo[6.4.0]dodecane, 10-benzyl-8-methyl-1,10-diazabicyclo[7.4.0]tridecane, 11-benzyl-1,11-diazabicyclo[8.4.0]tetradecane, 8-(2′-chlorobenzyl)-1,8-diazabicyclo[5.4.0]undecane, 8-(2′,6′-dichlorobenzyl)-1,8-diazabicyclo[5.4.0]undecane, 4-(diazabicyclo[4.3.0]nonanylmethyl)-1,1′-biphenyl, 4,4′-bis(diazabicyclo[4.3.0]nonanylmethyl)-11′-biphenyl, 5-benzyl-2-methyl-1,5-diazabicyclo[4.3.0]nonane, 5-benzyl-7-methyl-1,5,7-triazabicyclo[4.4.0]decane, and combinations thereof.

An example of a photolatent base is available from BASF under the trade designation “CGI-90”, which is reported to be 5-benzyl-1,5-diazabicyclo[4.3.0]nonane (see, e.g., WO 2014/176490 (Knapp et al.)), which generates 1,5-diazabicyclo[4.3.0]non-5-ene (DBN) upon exposure to radiation (see, e.g., US2013/0345389 (Cai et al), 2-benzyl-1-(3,5-dimethoxyphenyl)-2-(dimethylamino)butan-1-one available from BASF under the trade designation CGI 277, p-(Ethylthio)phenyl methylcarbamate and 6-Nitroveratryl chloroformate diethyl amine.

When using a photolatent base, the mixture comprised of the blocked 1,1-dicarbonyl substituted alkene may also include a photosensitizer. Photosensitizers are compounds when used in conjunction with a photolatent base improve or allow the accelerates the activation of the latent anionic initiator or allows for the activation at longer wavelengths than the absorbance of the photolatent base. A photosensitizer may be a compound having an absorption spectrum that overlaps or closely matches the emission spectrum of the radiation source to be used and that can, for example, improve the energy transfer to the photolatent base. Exemplary classes of photosensitizers include aromatic carbonyl compounds, for example benzophenone, thioxanthone, anthraquinone and 3-acylcoumarin derivatives or dyes such as eosine, rhodamine and erythrosine dyes. Additional exemplary photoinitiators include: thioxanthones, such as thioxanthone, 2-isopropylthioxanthone, 2-chlorothioxanthone, 1-chloro-4-propoxythioxanthone, 2-dodecylthioxanthone, 2,4-diethylthioxanthone, 2,4-dimethylthioxanthone, 1-methoxycarbonylthioxanthone, 2-ethoxycarbonylthioxanthone, 3-(2-methoxyethoxycarbonyl)-thioxanthone, 4-butoxycarbonylthioxanthone, 3-butoxycarbonyl-7-methylthioxanthone, 1-cyano-3-chlorothioxanthone, 1-ethoxycarbonyl-3-chlorothioxanthone, 1-ethoxycarbonyl-3-ethoxythioxanthone, 1-ethoxycarbonyl-3-aminothioxanthone, 1-ethoxycarbonyl-3-phenylsulfurylthioxanthone, 3,4-di-[2-(2-methoxyethoxy)ethoxycarbonyl]-thioxanthone, 1,3-dimethyl-2-hydroxy-9H-thioxanthen-9-one 2-ethylhexylether, 1-ethoxycarbonyl-3-(1-methyl-1-morpholinoethyl)-thioxanthone, 2-methyl-6-dimethoxymethyl-thioxanthone, 2-methyl-6-(1,1-dimethoxybenzyl)-thioxanthone, 2-morpholinomethylthioxanthone, 2-methyl-6-morpholinomethylthioxanthone, N-allylthioxanthone-3,4-dicarboximide, N-octylthioxanthone-3,4-dicarboximide, N-(1,1,3,3-tetramethylbutyl)-thioxanthone-3,4-dicarboximide, 1-phenoxythioxanthone, 6-ethoxycarbonyl-2-methoxythioxanthone, 6-ethoxycarbonyl-2-methylthioxanthone, thioxanthone-2-carboxylic acid polyethyleneglycol ester, 2-hydroxy-3-(3,4-dimethyl-9-oxo-9H-thioxanthon-2-yloxy)-N,N,N-trimethyl-1-propanaminium chloride; 2. Benzophenones, such as benzophenone, 4-phenyl benzophenone, 4-methoxy benzophenone, 4,4′-dimethoxy benzophenone, 4,4′-dimethyl benzophenone, 4,4′-dichlorobenzophenone 4,4′-bis(dimethylamino)-benzophenone, 4,4′-bis(diethylamino)benzophenone, 4,4′-bis(methylethylamino)benzophen-one, 4,4′-bis(p-isopropylphenoxy)benzophenone, 4-methyl benzophenone, 2,4,6-trimethyl-benzophenone, 4-(4-methylthiophenyl)-benzophenone, 3,3′-dimethyl-4-methoxy benzophenone, methyl-2-benzoylbenzoate, 4-(2-hydroxyethylthio)-benzophenone, 4-(4-tolylthio)-benzophenone, 1-[4-(4-benzoyl-phenylsulfanyl)-phenyl]-2-methyl-2-(toluene-4-sulfonyl)-propan-1-one, 4-benzoyl-N,N,N-trimethylbenzenemethanaminium chloride, 2-hydroxy-3-(4-benzoylphenoxy)-N,N,N-trimethyl-1-propanaminium chloride monohydrate, 4-(13-acryloyl-1,4,7,10,13-pentaoxamidecyl)-benzophenone, 4-benzoyl-N, N-dimethyl-N-[2-(1-oxo-2-propenyl)oxy]ethyl-benzenemethanaminium chloride; Coumarins, such as Coumarin 1, Coumarin 2, Coumarin 6, Coumarin 7, Coumarin 30, Coumarin 102, Coumarin 106, Coumarin 138, Coumarin 152, Coumarin 153, Coumarin 307, Coumarin 314, Coumarin 314T, Coumarin 334, Coumarin 337, Coumarin 500, 3-benzoyl coumarin, 3-benzoyl-7-methoxycoumarin, 3-benzoyl-5,7-dimethoxycoumarin, 3-benzoyl-5,7-dipropoxycoumarin, 3-benzoyl-6,8-dichlorocoumarin, 3-benzoyl-6-chloro-coumarin, 3,3′-carbonyl-bis[5,7-di(propoxy)coumarin], 3,3′-carbonyl-bis(7-methoxycoumarin), 3,3′-carbonyl-bis(7-diethylamino-coumarin), 3-isobutyroylcoumarin, 3-benzoyl-5,7-dimethoxy-coumarin, 3-benzoyl-5,7-diethoxy-coumarin, 3-benzoyl-5,7-dibutoxycoumarin, 3-benzoyl-5,7-di(methoxyethoxy)-coumarin, 3-benzoyl-5,7-di(allyloxy)coumarin, 3-benzoyl-7-dimethylaminocoumarin, 3-benzoyl-7-diethylaminocoumarin, 3-isobutyroyl-7-dimethylaminocoumarin, 5,7-dimethoxy-3-(1-naphthoyl)-coumarin, 5,7-diethoxy-3-(1-naphthoyl)-coumarin, 3-benzoylbenzo[f]coumarin, 7-diethylamino-3-thienoylcoumarin, 3-(4-cyanobenzoyl)-5,7-dimethoxycoumarin, 3-(4-cyanobenzoyl)-5,7-dipropoxycoumarin, 7-dimethylamino-3-phenylcoumarin, 7-diethylamino-3-phenylcoumarin, the coumarin derivatives disclosed in JP 09-179299-A and JP 09-325209-A, for example 7-[{4-chloro-6-(diethylamino)-S-triazine-2-yl}amino]-3-phenylcoumarin; 4. 3-(aroylmethylene)-thiazolines, such as 3-methyl-2-benzoylmethylene-p-naphthothiazoline, 3-methyl-2-benzoylmethylene-benzothiazoline, 3-ethyl-2-propionylmethylene-p-naphthothiazoline; Rhodanines, such as 4-dimethylaminobenzalrhodanine, 4-diethylaminobenzalrhodanine, 3-ethyl-5-(3-octyl-2-benzothiazolinylidene)-rhodanine; other Compounds, such as acetophenone, 3-methoxyacetophenone, 4-phenylacetophenone, benzil, 4,4′-bis(dimethylamino)benzil, 2-acetylnaphthalene, 2-naphthaldehyde, dansyl acid derivatives, 9,10-anthraquinone, anthracene, pyrene, aminopyrene, perylene, phenanthrene, phenanthrenequinone, 9-fluorenone, dibenzosuberone, curcumin, xanthone, thiomichler's ketone, α-(4-dimethylaminobenzylidene) ketones, e.g. 2,5-bis(4-diethylaminobenzylidene)cyclopentanone, 2-(4-dimethylamino-benzylidene)-indan-1-one, 3-(4-dimethylamino-phenyl)-1-indan-5-yl-propenone, 3-phenylthiophthalimide, N-methyl-3,5-di(ethylthio)-phthalimide, N-methyl-3,5-di(ethylthio)phthalimide, phenothiazine, methylphenothiazine, amines, e.g. N-phenylglycine, ethyl 4-dimethylaminobenzoate, butoxyethyl 4-dimethylaminobenzoate, 4-dimethylaminoacetophenone, triethanolamine, methyldiethanolamine, dimethylaminoethanol, 2-(dimethylamino)ethyl benzoate, poly(propylenegylcol)-4-(dimethylamino) benzoate. The weight ratio of photolatent anioinic initiators (e.g., base) to the weight of photosensitizers may be range from 0.5/1 to 10/1 or 1/1 to 5/1.

Anionic or free radical polymerization stabilizers may be present in sufficient amount to prevent premature polymerization of the blocked 1,1-dicarbonyl substituted alkene or be used to adjust the ratio of the unblocked alkenes that undergo addition polymerization or Michael addition crosslinking. Preferably, the anionic polymerization stabilizers such as methane sulfonic acid are present in an amount of about 0.1 part per million or greater based on the weight of the curable composition, more preferably about 1 part per million by weight or greater and most preferably about 5 parts per million by weight or greater. The anionic polymerization stabilizers may be present in an amount of about 1000 parts per million by weight or less based on the weight of the composition, more preferably about 500 parts per million by weight or less and most preferably about 100 parts per million by weight or less.

The blocked 1,1-dicarbonyl substituted alkene may comprise one or more free radical stabilizers. The one or more free radical stabilizers may be present in sufficient amount to prevent undesired addition polymerization as described in the previous paragraph. The free radical polymerization stabilizers may be present in an amount of about 10 ppm or less based on the weight of the total composition (the 1,1-dicarbonyl substituted alkene mixed with other ingredients) or the 1,1-dicarbonyl substituted alkene itself whether mixed with other ingredients or not, about 100 ppm by weight or greater, or about 1000 ppm by weight or greater. The free radical polymerization stabilizers may be present in an amount of about 10,000 ppm by weight or less based on the weight of the total composition, about 8000 ppm by weight or less, or about 5000 ppm by weight or less. The free radical inhibitors that may be used include: tocopherol (e.g., including vitamin E), 4-tert-Butylpyrocatechol; tert-Butylhydroquinone; 1,4-Benzoquinone; 6-tert-Butyl-2,4-xylenol; 2-tert-Butyl-1,4-benzoquinone; 2,6-Di-tert-butyl-p-cresol; 2,6-Di-tert-butylphenol; Hydroquinone; 4-Methoxyphenol; Phenothiazine; 2,2′-methylenebis(6-tert-butyl-4-methylphenol) or a combination thereof. Free radical stabilizers preferably include phenolic compounds (e.g., 4-methoxyphenol, mono methyl ether of hydroquinone (“MeHQ”) butylated hydroxytoluene (“BHT”)). Stabilizer packages for 1,1-disubstituted alkenes are disclosed in U.S. Pat. Nos. 8,609,885 and 8,884,051, each incorporated by reference. Additional free radical polymerization inhibitors are disclosed in U.S. Pat. No. 6,458,956 and are hereby incorporated by reference.

The blocked 1,1-dicarbonyl substituted alkene may contain a filler in certain embodiments such as printing dyes or additive manufacturing techniques such as polyjetting and inkjetting. Examples of filler include talc, wollastonite, mica, clay, montmorillonite, smectite, kaolin, calcium carbonate, glass fibers, glass beads, glass balloons, glass milled fibers, glass flakes, carbon fibers, carbon flakes, carbon beads, carbon milled fibers, metal flakes, metal fibers, metal coated glass fibers, metal coated carbon fibers, metal coated glass flakes, silica, other ceramic particles, ceramic fibers, ceramic balloons, aramid particles, aramid fibers, polyacrylate fibers, graphite, and various whiskers such as potassium titanate whiskers, aluminum borate whiskers and basic magnesium sulfate whiskers. The fillers may be incorporated alone or in combination.

The blocked 1,1-dicarbonyl substituted alkene and polymers made therefrom may be used in any number of applications. Exemplary applications include adhesives, sealants, films, coatings, potting and encapsulating materials for electronics, resins, molded articles, and the like.

To form the polymer or article from the blocked 1,1-dicarbonyl substituted alkene, the blocked 1,1-dicarbonyl substituted alkene is provided and each ingredient desired is mixed with the blocked 1,1-dicarbonyl substituted alkene to form a mixed composition. In an embodiment, the blocked 1,1-dicarbonyl substituted alkene is provided in at least two separate components prior to mixing as previously described. Desirably, blocked 1,1-dicarbonyl substituted alkene is provided in a first component and if desired an anionic initiator or multifunctional crosslinking MAD compound is provided in another component. The composition may be provided in a singular container having a plurality of chambers that separates each of the components from reacting prior to mixing of the component. The components may be dispensed through a common orifice causing each of the ingredients of the composition to mix. To aid the mixing through the orifice, a static or dynamic mixer may be employed. Illustratively, the blocked 1,1-dicarbonyl substituted alkene is mixed in a solvent or dispersed in a liquid (e.g., water) with any other desired ingredients, which then may be disposed on a substrate (e.g., coating, adhesive, caulk or the like).

In another embodiment, the blocked 1,1-dicarbonyl substituted alkene is provided in one component or container together, wherein the anionic initiator is latent as described previously. The ingredients in the composition may be dispensed under sufficient mixing (mechanical force) such that the latent anionic initiator is activated and upon unblocking of the blocked 1,1-dicarbonyl substituted alkene, it polymerizes forming the desired polymer or article. Alternatively, the blocked 1,1-dicarbonyl substituted alkene mixed with other ingredients as desired may be dispensed and then subjected to heating or irradiating to unblock the blocked 1,1-dicarbonyl substituted alkene and initiate polymerization.

When a photolatent anionic initiator (e.g., photolatent base) is used, the radiation source may be any suitable one such as those known in the art. Illustratively, the radiation is UV and the UV sources may be any suitable device such as those known in the art and include, for example, commercially available UV light emitting diodes (LEDs) and mercury lamps with or without filters.

In an embodiment a substrate is coated by the blocked 1,1-dicarbonyl substituted alkene forming an article with polymer formed from the unblocked 1,1-dicarbonyl substituted alkene adhered to the substrate. In another embodiment, the blocked 1,1-dicarbonyl substituted alkene is interposed on two or more substrates and polymerized to adhere the two or more substrates together. In another embodiment, after the blocked 1,1-dicarbonyl substituted alkene and any other ingredients may be dispensed, cast or injected into a mold and allowed to cure or react to form a shaped article. The dispensing to form a coated article or article comprised of two substrates adhered together by the composition's polymer reaction product may be any suitable dispensing method such as those known in the art (e.g., spraying, painting, caulk gunning, extruding and the like). The substrates may be any suitable substrate such as a ceramic, metal, metalloid, glass, plastic, wood, a composite of any of the aforementioned, or combination thereof.

The polymer that is formed may allow for the deconstructing of the formed article without decomposition of the underlying 1,1-dicarbonyl substituted alkene. For example, the article may be deconstructed when the polymer has sufficient number of the Michael addition crosslinks such that upon heating to the deconstruction temperature the Michael addition crosslinks revert to alkenes allowing for the deconstruction of the article. Illustratively, the method allows the separation of adhered substrates that then may be easily reused or recycled without damage to the under lying substrate. The deconstruction temperature may be any temperature such as described above for the polymerization temperature. Typically, a sufficient amount of Michael addition crosslinks is at least about 10%, 20%, 40%, or even 50% to about 99% by mole of the alkene bonds of the underlying blocked 1,1-dicarbonyl substituted alkene are Michael addition crosslinks. The heating may be performed in any atmosphere (e.g., inert gas, nitrogen, air, dry air, or combination thereof) with air being convenient. The atmosphere may also be at a reduced pressure). The heating may be performed in the presence of a strong acid or stabilizer to impede undesired addition polymerization of the alkenes that become unblocked during the deconstruction.

### ILLUSTRATIVE EMBODIMENTS

Embodiment 1. A method to form a blocked 1,1-dicarbonyl substituted alkene comprising:

mixing a 1,1-dicarbonyl substituted alkene with a blocking Michael addition donor compound, to form a reaction mixture, and
providing a reaction temperature to the reaction mixture to react the blocking Michael addition donor with an alkene of the 1,1-dicarbonyl substituted alkene to form the blocked 1,1-dicarbonyl substituted alkene.

Embodiment 2. The method of Embodiment 1, wherein the blocking Michael addition donor compound is comprised of an amine, alcohol, carboxylic acid, or thiol.

Embodiment 3. The method of Embodiment 2, wherein the blocking Michael addition donor compound is comprised of a monofunctional blocking Michael addition donor compound.

Embodiment 4. The method of any one of Embodiments 1 to 3, wherein the blocking Michael addition donor compound is comprised of an alcohol, thiol or mixture t.

Embodiment 5. The method of any one of the preceding Embodiments, wherein the blocking Michael addition donor compound is a mixture of alcohols and thiols.

Embodiment 6. The method of any one of the preceding Embodiments, wherein the blocking Michael addition donor compound has a weight molecular weight average of at most 500 g/moles.

Embodiment 7. The method of any one of the previous Embodiments, wherein the blocking Michael addition donor compound has a molecular weight average of at most about 200 g/moles.

Embodiment 8. The method any one of the preceding Embodiments, wherein the blocking Michael addition donor compound has a weight average molecular weight of at most 150 g/moles.

Embodiment 9. The method of any one of the preceding Embodiments, wherein the blocking Michael addition donor compound is an alcohol or thiol having a molecular weight of at most 150 g/moles.

Embodiment 10. The method of Embodiment 9, wherein the alcohol is methanol, ethanol, propanol, butanol, pentanol, hexanol, heptanol, octanol or mixture thereof.

Embodiment 11. The method of Embodiment 9, wherein the thiol is butanethiol, pentanethiol, hexanethiol, heptanethiol, octanethiol, thioterpineol, thioacetic acid, mercaptoethanol or mixture thereof.

Embodiment 12. The method of any one of the preceding Embodiments, wherein the reaction temperature is above about 20° C. to about 150° C.

Embodiment 13. The method of any one of the preceding Embodiments, wherein the reaction is performed by refluxing the blocking Michael addition donor compound.

Embodiment 14. The method of any one of the preceding Embodiments 1 to 12, wherein the reaction is performed at a temperature below the boiling point of the Michael addition donor compound.

Embodiment 15. The method of any one of the preceding Embodiments, wherein the reaction is performed for a reaction time sufficient to block at least about 50 mole percent of the alkenes of the 1,1-dicarbonyl substituted alkene.

Embodiment 16. The method of any one of the preceding Embodiments, wherein the reaction time is from several minutes to 24 hours.

Embodiment 17. The method of any one of the preceding Embodiments, wherein the reaction mixture is further comprised of one or more of a strong acid.

Embodiment 18. The method of Embodiment 17, wherein the strong acid has a pKa of about 3 to −12.

Embodiment 19. The method of Embodiment 18, wherein the strong acid is a sulfonic acid or a mixture of sulfonic acids or is sulfuric acid

Embodiment 20. The method of any one of the preceding Embodiments, wherein the 1,1-dicarbonyl substituted alkene is represented by:

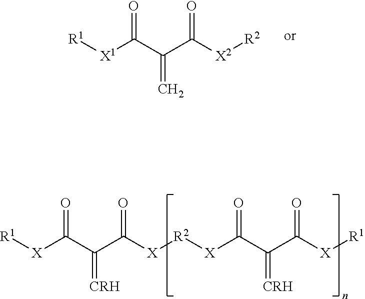

wherein X, X¹ and X² are an oxygen atom or a direct bond, and where R¹ and R² are each hydrocarbyl groups having from 1 to 30 carbons and R is hydrogen or a hydrocarbyl group having from 1 to 30 carbons, so long as at least one R is hydrogen.

Embodiment 21. The method of Embodiment 20, wherein X is an oxygen atom and R² is the residue of a polyol.

Embodiment 22. The method of any one of preceding Embodiments, wherein the 1,1-dicarbonyl substituted alkene is comprised of one or more of methyl propyl methylene malonate, dihexyl methylene malonate, di-isopropyl methylene malonate, butyl methyl methylene malonate, ethoxyethyl ethyl methylene malonate, methoxyethyl methyl methylene malonate, hexyl methyl methylene malonate, dipentyl methylene malonate, ethyl pentyl methylene malonate, methyl pentyl methylene malonate, ethyl ethylmethoxy methylene malonate, ethoxyethyl methyl methylene malonate, butyl ethyl methylene malonate, dibutyl methylene malonate, diethyl methylene malonate (DEMM), diethoxy ethyl methylene malonate, dimethyl methylene malonate, di-N-propyl methylene malonate, ethyl hexyl methylene malonate, methyl fenchyl methylene malonate, ethyl fenchyl methylene malonate, 2 phenylpropyl ethyl methylene malonate, 3 phenylpropyl ethyl methylene malonate, ethyl cyclohexyl methylene malonate, and dimethoxy ethyl methylene malonate.

Embodiment 23. The method of any one of the preceding Embodiments, wherein the 1,1-dicarbonyl substituted alkene is the reaction product of the transesterification of a polyol and diethyl methylene malonate.

Embodiment 24. The method of any one of the preceding Embodiments, wherein the 1,1-dicarbonyl substituted alkene has an average alkene functionality of greater than one to about 25.

Embodiment 25. The method of Embodiment 24, wherein the average alkene functionality is about 2 to 20.

Embodiment 26. A blocked 1,1-dicarbonyl substituted alkene comprised of the reaction product of a 1,1-dicarbonyl substituted alkene and monofunctional blocking Michael addition donor compound.

Embodiment 27. The blocked 1,1-dicarbonyl substituted alkene of Embodiment 26, wherein the blocking Michael addition donor compound has a molecular weight of at most about 500 g/moles.

Embodiment 28. The blocked 1,1-dicarbonyl substituted alkene of either Embodiment 26 or 27 is further comprised of one or more of a strong acid.

Embodiment 29. The blocked 1,1-dicarbonyl substituted alkene of anyone of Embodiments 26 to 28, wherein the blocked 1,1-dicarbonyl substituted alkene is comprised of alkenes that have not been blocked.

Embodiment 30. The blocked 1,1-dicarbonyl substituted alkene of Embodiment 29, wherein the blocked 1,1-dicarbonyl substituted alkene has an average alkene functionality of greater than 0 to about 2.

Embodiment 31. The blocked 1,1-dicarbonyl substituted alkene of Embodiment 30, wherein the average alkene functionality is greater than 0 to at most 1.

Embodiment 32. The blocked 1,1-dicarbonyl substituted alkene of anyone of Embodiments 26 to 31, wherein at least about 50 mole percent of the alkenes of the 1,1-dicarbonyl substituted alkene have been blocked by the monofunctional blocking Michael addition donor compound.

Embodiment 33. A method of forming a polymer comprising,
(i) providing the blocked 1,1-dicarbonyl substituted alkene of any one of the preceding Embodiments, and
(ii) heating the blocked 1,1-dicarbonyl substituted alkene to a polymerization temperature, wherein at least a portion of the blocked alkenes are unblocked and at least a portion of said alkenes undergo addition polymerization to form the polymer.

Embodiment 34. The method of Embodiment 33, wherein upon heating to the polymerization temperature at least a portion of the blocking Michael addition donor compound is liberated and removed during the heating.

Embodiment 35. The method of Embodiment 34, wherein the blocking Michael addition donor compound is monofunctional.

Embodiment 36. The method of Embodiment 35, wherein the blocking Michael addition donor compound is an alcohol or thiol.

Embodiment 37. The method of any one of Embodiments 33 to 36 further comprising mixing a crosslinking multifunctional Michael addition donor compound with the 1,1-dicarbonyl substituted alkene prior to or during the heating to the polymerization temperature.

Embodiment 38. The method of Embodiment 37, wherein the polymer is comprised of crosslinking through the cross-linking multifunctional Michael addition donor compound and addition polymerized alkenes.

Embodiment 39. The method of either Embodiment 37 or 38, wherein the crosslinking multifunctional Michael addition donor compound has a molecular weight that is greater than the blocking Michael addition donor compound.

Embodiment 40. The method of any one of Embodiments 33 to 39 further comprising the addition of an addition polymerization catalyst.

Embodiment 41. The method of Embodiment 40, wherein the addition polymerization catalyst is an anionic polymerization initiator.

Embodiment 42. The method of Embodiment 41, wherein anionic polymerization initiator is comprised of a latent base.

Embodiment 43. The method of Embodiment 41 wherein the anionic polymerization initiator is comprised of an amine.

Embodiment 44. The method of Embodiment 43, wherein the amine is a tertiary amine.

Embodiment 45. The method of any one of the preceding Embodiments, wherein the blocked 1,1-dicarbonyl substituted alkene is mixed with one or more of a filler, dye, stabilizer, plasticizer or lubricant.

Embodiment 46. The method of any one of Embodiments 33 to 45, wherein the blocked 1,1-dicarbonyl substituted alkene is contacted with a first substrate forming a polymer that is adhered to the substrate.

Embodiment 47. The method of Embodiment 46, wherein a second substrate is brought into contact with the blocked 1,1-dicarbonyl substituted alkene on the first substrate such that the polymer adheres to the first and second substrate.

Embodiment 48. The method of either Embodiment 46 or 47, wherein the substrate or another substrate is a ceramic, metal, glass, plastic, wood, a composite of any of the aforementioned, or combination thereof.

Embodiment 49. An article comprised of the polymer of any one of Embodiments 33 to 45.

Embodiment 50. The article of Embodiment 49, wherein the polymer is adhered to a substrate

Embodiment 51. The article of Embodiment 50, wherein the article is the substrate coated by the polymer.

Embodiment 52. The article of Embodiment 51, wherein the article is two or more substrates adhered together by the polymer.

Embodiment 53. The article of either Embodiment 51 or 52, wherein the polymer is comprised of sufficient number of Michael addition crosslinks such that upon heating to a deconstruction temperature, the Michael addition crosslinks revert to alkenes allowing for the deconstruction of the article.

Embodiment 54. The article of 53, wherein the polymer has a molar ratio of addition polymerized alkenes/Michael added alkenes of about 10 to about 0.1.

Embodiment 55. The article of any one of Embodiments 50 to 54, wherein the deconstruction temperature is below a temperature where the polymer and the substrates decompose.

Embodiment 56. The article of any one of Embodiments 53 to 55, wherein the heating is in air.

Embodiment 57. A method of deconstructing an article comprising,
(i) providing an article comprising a polymer of any of Embodiment 33 to 45,
(ii) heating the article to a deconstruction temperature, wherein the polymer is comprised of sufficient number of Michael addition crosslinks such that upon heating to the deconstruction temperature, the Michael addition crosslinks revert to alkenes allowing for the deconstruction of the article, and
(iii) deconstructing the article.

Embodiment 58. The method of Embodiment 57, wherein the deconstruction temperature is below a temperature where the polymer and the substrate decompose.

Embodiment 59. The method of either Embodiment 57 or 58, wherein the heating is performed in air.

Embodiment 60. The method any one of Embodiment 57 to 59, wherein the polymer has a molar ratio of addition polymerized alkenes/Michael added alkenes of about 10 to about 0.1.

Embodiment 61. The method any one of Embodiment 56 to 59, wherein the heating performed in the presence of a strong acid or stabilizer.

Embodiment 62. The method of any one of Embodiments 57 to 61, wherein the polymer is adhered to one or more substrates.

Embodiment 63. A polymer comprised of a reaction product of a composition comprising a 1,1-dicarbonyl substituted carbonyl, monofunctional blocking Michael addition donor compound and a multifunctional Michael addition donor compound.

Embodiment 64. The article of Embodiment 49, wherein the article is an additive manufactured article comprised of at two layers of the polymer adhered together.

### EXAMPLES

The following examples are provided to illustrate the curable compositions and the polymers formed from them, but are not intended to limit the scope thereof. All parts and percentages are by weight unless otherwise noted. Table 1 shows the ingredients used in the examples and comparative examples.

Example A: A blocked 1,1-dicarbonyl substituted alkene compound (referred as polyester Michael Adduct or PEMA below) was synthesized as follows. All equipment was passivated with 1% solution of methanesulfonic acid (MSA) in tetrahydrofuran (THF), followed by 3 rinses with dry acetone and air drying. A 4 necked round bottom 1 L flask was equipped with stainless steel stirrer with stainless steel crescent stirring blade using an overhead IKA motor, a condenser, thermocouples, JKem temperature controller, and heating mantle. Methylene malonate polyester (here and below mentioned as BDPES) prepared from diethyl methylene malonate and 1,4-butanediol (250.4 g, Sirrus Inc. under Forza B5200 XP name) was charged, followed by Isopropanol (262 g, Sigma) and MSA (0.745 g, from Sigma). Stirring started at 100 rpm and was maintained throughout the reaction. Initial reaction temperature was 18.6° C. Heating began and in 17 minutes the reaction temperature reached 77.6° C. Heating continued with the set point of 85° C. The reaction mixture reached 82.3° C. in 29 minutes from the start and at 84.5° C. reflux began. The reaction mixture was held above 80° C. for a total of about 13 hours. After that, excess isopropanol was removed using a rotary evaporator under vacuum at 31° C.

The double bond conversion was calculated from quantitative NMR analysis of the reaction product to be 84.8%. 283.2 g of light brown color liquid was isolated. Molecular weight was measured by GPC using polymethylmethacrylate (PMMA) as the standard (for the main peak area constituting 89.2% of the total chromatogram): Mn=1056, Mw=1654, PD=1.57.

Examples 1-3 and Comparative Examples 1-3: Example coating compositions incorporating PEMA and comparative coating compositions incorporating BDPES (available as Forza B5200 XP from Sirrus Inc.) were formulated by mixing the ingredients as shown in Table 1. The compositions were coated on cold rolled steel panels using Mayer rod #10 (from RD Specialities). The coated panels were then placed into an oven preheated to 205° C. The panels were held in the oven for 5 minutes. The metal temperature for the uncoated panel area ranged from 196° C. to 208° C. The coating properties were measured after the panels were removed from the oven and cooled to ambient temperature. Mandrel Bend was performed on a conical mandrel (Elcometer 1510) using ASTM D522-17 method. MEK double rubs were performed per ASTM D5402-93 method. Crosshatch adhesion was determined using ASTM D3359-09 procedure. The resistance to water was determined by immersing coated panels into 50° C. water for 1 hour: the coatings were then inspected for any damage and results were summarized in Table 1.

TABLE 1

| | | | | Comp. | Comp. | Comp. |
|---|---|---|---|---|---|---|
| Composition (% wt) | Example 1 | Example 2 | Example 3 | Ex. 1 | Ex. 2 | Ex. 3 |
| BDPES | | | | 99.70 | 19.90 | 99.70 |
| PEMA | 100.00 | 19.90 | 54.62 | | | |
| BYK-333 Additive | | | | 0.30 | | 0.30 |
| *Joncryl 935 Acrylic | | 46.40 | 29.41 | | 46.40 | |
| Polyol (recalculated | | | | | | |
| to 100% solids) | | | | | | |
| Methyl n-Amyl | | 19.90 | 12.61 | | 19.90 | |
| Ketone (solvent) | | | | | | |
| p-toluenesulfonic acid | | 0.70 | 0.84 | | 0.70 | |
| (catalyst) | | | | | | |
| Methyl ethyl ketone | | 13.10 | 2.52 | | 13.10 | |
| Total | 100.00 | 100.00 | 100.00 | 100.00 | 100.00 | 100.00 |
| Appearance | clear film | clear film | | clear film | clear film | clear film |
| | with | with slight | | | | with |
| | yellowing | yellowing | | | | yellow |
| | | | | | | tinge |
| Cured coating | 11-12 | 7-8 | 9-10 | 7-11 | 9-11 | 11-12 |
| thickness, mm | | | | | | |
| Mandrel Bend | no failure | no failure | 40 | no failure | no failure | no failure |
| (failure length, mm) | | | | | | |
| (ASTM D522) | | | | | | |
| MEK Double Rubs | >200 | 0 | 80 | 50 | 10 | >200 |
| (ASTM 5402-93) | | | | | | |
| Crosshatch Adhesion | 5B | 4B | 0 | 3B | 5B | 4B |
| (ASTM D3359-09) | | | | | | |
| 20/60 Gloss | 110/124.4 | | | | | 84/118 |
| (ASTM D523-08) | | | | | | |
| Water immersion, | no damage | | | | | loss of |
| 50° C. 1hr | | | | | | adhesion |

*Joncryl 935 was used as 70% solids in methyl n-amyl ketone (MAK)

What is claimed is:

1. A method to form a blocked 1,1-dicarbonyl substituted alkene comprising:
(i) mixing a 1,1-dicarbonyl substituted alkene with a blocking Michael addition donor compound, to form a reaction mixture, and
(ii) providing a reaction temperature to the reaction mixture to react the blocking Michael addition donor with an alkene of the 1,1-dicarbonyl substituted alkene to form the blocked 1,1-dicarbonyl substituted alkene.

2. The method of claim 1, wherein the blocking Michael addition donor compound is comprised of an alcohol, thiol or mixture t.

3. The method of claim 2, wherein the blocking Michael addition donor compound has a weight molecular weight average of at most 500 g/moles.

4. The method of claim 1, wherein the blocking Michael addition donor compound is methanol, ethanol, propanol, butanol, pentanol, hexanol, heptanol, octanol, butanethiol, pentanethiol, hexanethiol, heptanethiol, octanethiol, thioterpineol, thioacetic acid, mercaptoethanol or mixture thereof.

5. The method of claim 1, wherein the reaction mixture is further comprised of one or more of a strong acid.

6. The method of claim 1, wherein the 1,1-dicarbonyl substituted alkene is represented by: 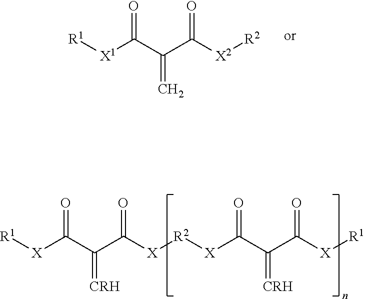
wherein X, X¹ and X² are an oxygen atom or a direct bond, and where R¹ and R² are each hydrocarbyl groups having from 1 to 30 carbons and R is hydrogen or a hydrocarbyl group having from 1 to 30 carbons, so long as at least one R is hydrogen.

7. The method of claim 1, wherein the 1,1-dicarbonyl substituted alkene has an average alkene functionality of greater than one to about 25.

8. A blocked 1,1-dicarbonyl substituted alkene comprised of the reaction product of a 1,1-dicarbonyl substituted alkene and monofunctional blocking Michael addition donor compound.

9. The blocked 1,1-dicarbonyl substituted alkene of claim 8, wherein the monofunctional blocking Michael addition donor compound has a molecular weight of at most about 500 g/moles.

10. The blocked 1,1-dicarbonyl substituted alkene of claim 8 further comprising a strong acid.

11. The blocked 1,1-dicarbonyl substituted alkene of claim 8, wherein the blocked 1,1-dicarbonyl substituted alkene is comprised of alkenes that have not been blocked.

12. The blocked 1,1-dicarbonyl substituted alkene of claim 8, wherein the average alkene functionality is greater than 0 to at most 1.

13. The blocked 1,1-dicarbonyl substituted alkene of claim 8, wherein at least about 50 mole percent of the alkenes of the 1,1-dicarbonyl substituted alkene have been blocked by the monofunctional blocking Michael addition donor compound.

14. A method of forming a polymer comprising,
(i) providing a blocked 1,1-dicarbonyl substituted alkene comprised of the reaction product of a 1,1-dicarbonyl substituted alkene and monofunctional blocking Michael addition donor compound, and
(ii) heating the blocked 1,1-dicarbonyl substituted alkene to a polymerization temperature, wherein at least a portion of the blocked alkenes are unblocked and at least a portion of said alkenes undergo addition polymerization to form the polymer.

15. The method of claim 14, wherein upon heating to the polymerization temperature at least a portion of the blocking Michael addition donor compound is liberated and removed during the heating.

16. The method of claim 14, wherein the blocking Michael addition donor compound is an alcohol, thiol or mixture thereof.

17. The method of claim 15 further comprising mixing a crosslinking multifunctional Michael addition donor compound with the blocked 1,1-dicarbonyl substituted alkene to or during the heating to the polymerization temperature.

18. The method of claim 17, wherein the polymer is comprised of crosslinking through the cross-linking multifunctional Michael addition donor compound and addition polymerized alkenes.

19. The method of claim 18, wherein the blocked 1,1-dicarbonyl substituted alkene is contacted with a first substrate forming a polymer that is adhered to the substrate.

20. The method of claim 19, wherein a second substrate is brought into contact with the blocked 1,1-dicarbonyl substituted alkene on the first substrate such that the polymer adheres to the first and second substrate.
